(19) United States
(12) Patent Application Publication
(10) Pub. No.: US 2003/0195770 A1
(43) Pub. Date: Oct. 16, 2003

(54) Medical data processing system

(75) Inventor: Kazuhisa Fukushima, Musashino-shi (JP)

Correspondence Address: ARMSTRONG,WESTERMAN & HATTORI, LLP, 1725 K STREET, NW, SUITE 1000, WASHINGTON, DC, 20006, US

(73) Assignee: Yokogawa Electric Corporation, Musashino-shi (JP)

(21) Appl. No.: 10/160,049

(22) Filed: Jun. 4, 2002

(30) Foreign Application Priority Data
Apr. 16, 2002 (JP) 2002-113088

(51) Int. Cl. G06F 17/60; G06F 19/00; G01N 33/48; G01N 33/50; G06F 7/00; G06F 17/00

(52) U.S. Cl. 705/2; 707/104.1; 702/19

(57) ABSTRACT

The present invention realizes a system which enables to smoothly and efficiently study and search for disease condition, disease names, treatment methods, medication methods and other information and to smoothly and efficiently follow the procedure of paying expenses necessary for the above process. It also provides a medical data processing system in which data analysis units for medical use are connected to a communications network, wherein a database is installed as a portal site which will be accessed by the data analysis units. The present invention comprises a content server having databases for storing disease condition, disease names, treatment methods, medication methods, and other information, an analysis server for correlating the comparison result with an individual patient as a diagnostic analysis and outputting personal information, and a billing/inventory control server for outputting fee information appropriate for the comparison result included in the personal information.

### BACKGROUND OF THE INVENTION

1. Field of the Invention

The present invention relates to a medical data processing system having disease condition information, disease name information, pathology information, diagnosis information, and treatment information as data in the medical field, and capable of quickly executing searches for optimum prescription information and other tasks.

The present invention further relates to a medical data processing system capable of quickly executing billing processes necessary for using the aforementioned data.

2. Description of the Prior Art

Biotechnology has advanced recently and a wide range of applied technologies will be employed in the medical field. Specifically, as shown in FIG. 1, the typical flow of processes in the field of medical services will be 1) diagnosis by doctors, 2) blood collection and enucleation of affected parts from patients, 3) preprocessing (chromosome/DNA/RNA/protein extraction), 4) chromosome/DNA/RNA/protein chip based detection (detection of specific chromosome/DNA/RNA/protein), 5) comparison between extracted chromosome/DNA/RNA/protein samples and the probes on the chromosome/DNA/RNA/protein chips by means of a reader to read the result of the comparison, and 6) analysis of the comparison result and subsequent data analysis. These processes are further followed by the process of studying and searching for disease condition, disease names, treatment methods, and medication methods using the diagnostic know-how resides on the Content server and/or partially resides on the Data analysis unit at site as shown in the FIGS. 2 and 3 as well as using the published literatures, papers, and references. The diagnostic know-how identifies and authorizies the relation between the chromosome/DNA/RNA/protein expression profiles patterns on the chromosome/DNA/RNA/protein chips and the disease condition, disease names, treatment methods, and medication methods. In the aforementioned flow of processes, respective blocks in the diagram of FIG. 1 may be processed as a series of processes by only one special institution. In most cases, however, each of the diagnostic know-how is developped by each individual medical sector such as a research institution, university, research laboratory, private-sector company. Thus, the processes are very often undertaken by a plurality of institutions.

Likewise, the diagnostic know-how and each database of literatures, papers and references to be used for the process of studying and searching for disease conditions, disease names, treatment methods, and medication methods is often separately located in a decentralized way by each individual medical sector such as a research institution, university, research laboratory, private-sector company, or literature publisher. Consequently, the procedure required for such study and search processes has been cumbersome.

Now assume that one has obtained results of studying and searching for the aforementioned cases, treatment methods and medication methods. Then, the next step is pay know-how fees, costs spent on the procedure, and so on, to the research institution, university, and private-sector company. The process of such payment varies from bank transfer to cash and credit card, depending on whether the recipient is a research institution, university, or private-sector company. Thus, the process has also been extremely cumbersome.

### SUMMARY OF THE INVENTION

The present invention is intended to eliminate such cumbersome work as discussed above. An object of the present invention is therefore to realize a system whereby it is possible to study and search for disease condition, disease names, treatment methods, and medication methods smoothly and efficiently by the diagnostic know-how in the centralized portal site and/or authorized partial copies of the diagnostic know-how resides in the Data analysis unit at site

Another object of the present invention is to realize a system whereby it is possible to smoothly and efficiently follow the procedure of paying expenses necessary for the aforementioned diagnostic know-how, study and search processes.

The present invention intended to achieve these objects is as follows:

(1) A medical data processing system in which data analysis units for medical use are connected to a communications network, wherein a database of at least one case, whose corresponding recipes are stored as data and therefore a plurality of recipe items is provided, is installed as a portal site to be accessed by the data analysis units.

(2) The medical data processing system of item 1, wherein the case is subdivided into subdivided disease condition so as to have a hierarchical structure.

(3) The medical data processing system of item 1 or 2, wherein the portal site comprises a billing system for requesting accesses to execute billing according to the state of use of the recipe items when access is made thereto.

(4) The medical data processing system of item 1, 2 or 3, wherein the recipe items are such that the corresponding recipes contained therein are linked to the location of storage of original data.

(5) The medical data processing system of item 1, 2 or 3, wherein the data analysis units gain access to the recipe items and download data contained therein.

(6) A medical data processing system in which data analysis units for medical use are connected to a communications network, comprising:

a content server having databases for storing disease condition, disease names, treatment methods, medication methods, and other information, as diagnosis protocols, in order to output the result of comparing the databases with data sent from the data analysis unit;

an analysis server for correlating the comparison result with an individual patient as a diagnostic analysis and outputting personal information; and

a billing/inventory control server for outputting fee information appropriate for the comparison result included in the personal information from a billing database and sending back payment request/inventory control information to the data analysis units by outputting inventory control information from an inventory/register database containing stock data on, for example, medicines and treatment equipment.

(7) The medical data processing system of item 6, wherein the data is the expression profiles analysis data of chromosome/DNA/RNA/protein or the cartridge/analyzer ID data thereof, the amount-of-expression analysis data of chromosome/DNA/RNA/protein or the cartridge/analyzer ID data thereof, or the expression profiles analysis data of SNPs or the cartridge/analyzer ID data thereof.

(8) The medical data processing system of item 6 or 7, wherein disease condition, disease names, treatment methods, medication methods, and other information appropriate for the expression profiles analysis data of chromosome/DNA/RNA/protein or the cartridge/analyzer ID data thereof, the amount-of-expression analysis data of chromosome/DNA/RNA/protein or the cartridge/analyzer ID data thereof, or the expression profiles analysis data of SNPs or the cartridge/analyzer ID data thereof, from the data analysis units are registered as new content (diagnosis protocols) in the content server.

(9) The medical data processing system of item 6, wherein the analysis server receives personal objective raw analysis data directly sent from the data analysis units as personal information, and stores the information.

(10) The medical data processing system of item 6, wherein the data analysis units are provided with an interface for combining at least one piece of information, among the comparison result from the content server, the personal information from the analysis server, and the payment request/inventory control information from the billing/inventory control server, with regular test data or regular diagnosis data.

### BRIEF DESCRIPTION OF THE DRAWINGS

FIG. 1 is a systematic block diagram explaining the progress of conventional medical diagnosis.

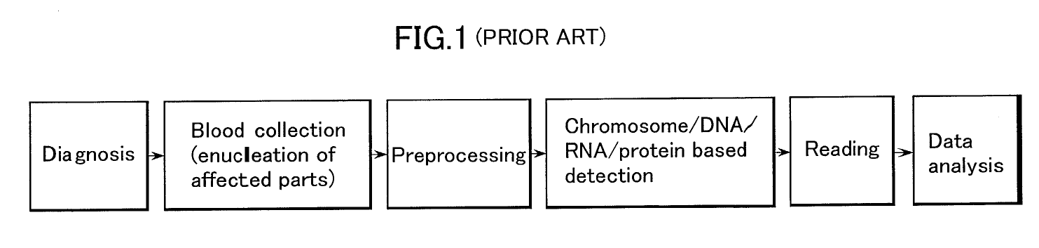

FIG. 2 is a block diagram showing the configuration of a medical data processing system according to the present invention.

FIG. 3 is another block diagram showing the configuration of a medical data processing system according to the present invention.

### DETAILED DESCRIPTION OF THE PREFERRED EMBODIMENTS

Now, modes of carrying out the present invention are described in detail by referring to the accompanying drawings.

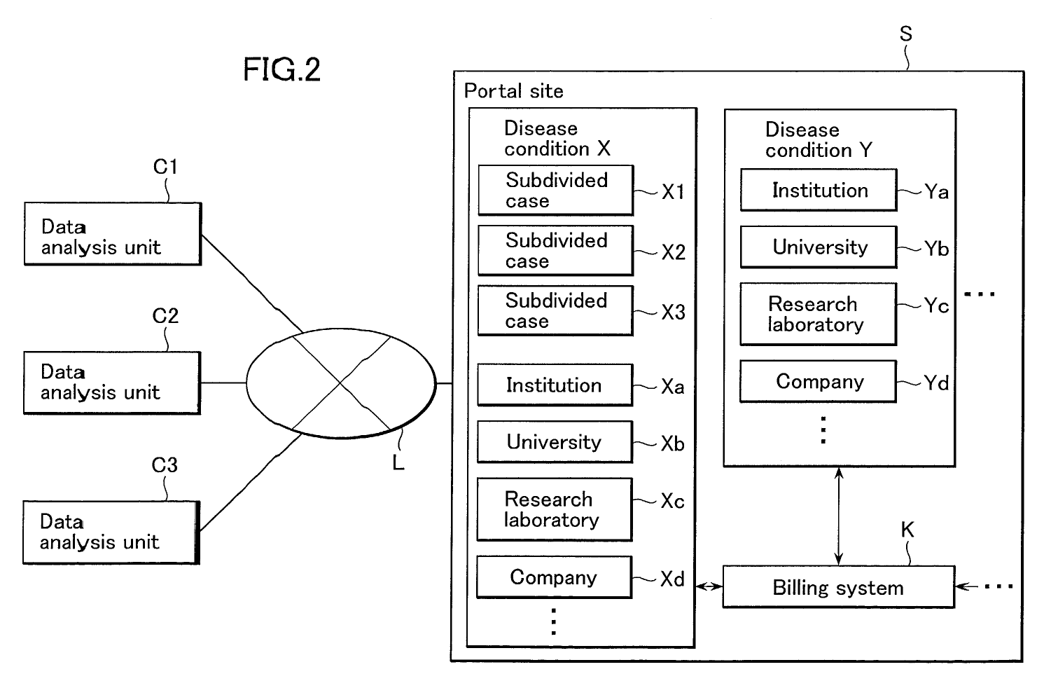

FIG. 2 is a block diagram showing the configuration of a medical data processing system according to the present invention.

Data analysis units C1, C2 and C3, for which it is a common practice to use small-scale computer systems, including regular personal computers, located within the reach of such users as hospitals, doctors, pharmacists, and medical consultants, are connected to a communications network L.

A portal site S connected to the communications network L serves as a so-called software bank which is built on the basis of research institutions, universities, research laboratories, private-sector companies, or literature publishers sporadically located in each district or outside Japan, as described earlier, and whereby literature, paper and reference names to be used for studying and searching for disease condition, disease names, treatment methods, and medication methods are virtually brought together at one location. Thus, the portal site S is a database that functions as an information source for collectively controlling the literature names, paper names, reference names, and other pieces of information.

More specifically, the portal site S is given an enumeration of, for example, dementia, cancer, diabetes, hypertension, asthma, and other diseases as case X, case Y, . . . . If any one case needs to be subdivided into such subclasses as types of cancer, then subdivided disease condition x1, x2, x3, . . . are established under case X so that case X has a hierarchical structure.

In such recipe items as each institution Xa, university Xb, research laboratory Xc, and company Xd, disease names, treatment methods, medication methods, and other information corresponding to case X and subdivided disease condition x1, x2, x3, . . . , in which each recipe item specializes, are stored as data. These disease names, treatment methods, medication methods, and other information exist on the side of data analysis units C1, C2 and C3, where it is possible to obtain desired data through the communications network L by means of keyword search or mouse clicking.

Likewise, in such recipe items as each institution Ya, university Yb, research laboratory Yc, and company Yd, disease names, treatment methods, medication methods, and other information corresponding to case Y, in which each recipe item specializes, are stored as data. These disease names, treatment methods, medication methods, and other information exist on the side of data analysis units C1, C2 and C3, where desired data can be obtained through the communications network L.

In such a system as described above, the process of comparing between an extracted chromosome/DNA/RNA/protein sample and a chromosome/DNA/RNA/protein chip with a reader to read the comparison result, and analyzing the comparison result is completed using the data analysis unit C1 (C2 or C3) located within the reach of such a user as a hospital or a doctor. If it is necessary to proceed to the process of studying and searching for disease condition, disease names, treatment methods, and medication methods, the user gains access to the portal site S through the communications network L, and selects the relevant case, case X or case Y. If access needs to be made to the subdivided case x1 (x2 or x3), the user first selects case X and then moves down to the lower layer to select a subdivided case.

In the next step, the user selects an arbitrary institution Xa, university Xb, research laboratory Xc, or company Xd by means of, for example, mouse clicking, according to case X or subdivided case x1, gains access to a desired recipe item, and obtains medication methods, treatment methods and other information corresponding to that case through the screen of or printouts from the data analysis unit accessible to the user.

Alternatively, the system may be configured so that each recipe item located on the side of the portal site S may be downloaded as data to the data analysis unit C1, C2 or C3.

In that case, each recipe item, such as the institution Xa, university Xb, research laboratory Xc, and company Xd, may be given a heading or a title representing the item′ specialization or regarding the item as a unique database. Consequently, it is possible to easily select or search for a desired recipe item.

Furthermore, a billing system K is installed in the portal site S so that when access is made to any of the recipe items Xa, Xb, Xc, Xd, . . . , Ya, Yb, Yc, Yd, . . . within the portal site S from the data analysis unit C1, C2 or C3, an appropriate fee is paid for that recipe item (for example, the institution that owns the original data) by means of, for example, credit card.

Thus, various databases of disease condition and other information that used to be separately located in different places are integrated into the portal site S. Consequently, it is possible to select and search for desired information from the data analysis unit C1, C2 or C3 within the reach of, for example, doctors, and quickly know the relevant medical prescription and treatment method given as recipe items.

Note that the recipe items Xa, Xb, Xc, Xd, . . . , Ya, Yb, Yc, Yd, . . . may be configured so that when clicked on, the user jumps to their respective linked homepages that introduce the institution, university, research laboratory, or company.

Furthermore, the system is provided with security functions since data from the data analysis unit C1, C2 or C3 is private.

As a summary of the discussion made heretofore, the following advantages are provided according to the present invention.

The medical data processing system according to the present invention is configured so that separately located databases of literatures, papers and references to be used for studying and searching for disease condition, disease names, treatment methods, and medication methods are integrated into one portal site and a billing system is provided therein as necessary. Consequently, it is possible to eliminate the cumbersome process of studying and searching for treatment methods, medication methods and other information for the case in question, thus realizing a system that allows the user to smoothly and efficiently study and search for such information.

Now, the present invention will be described further by referring to another embodiment.

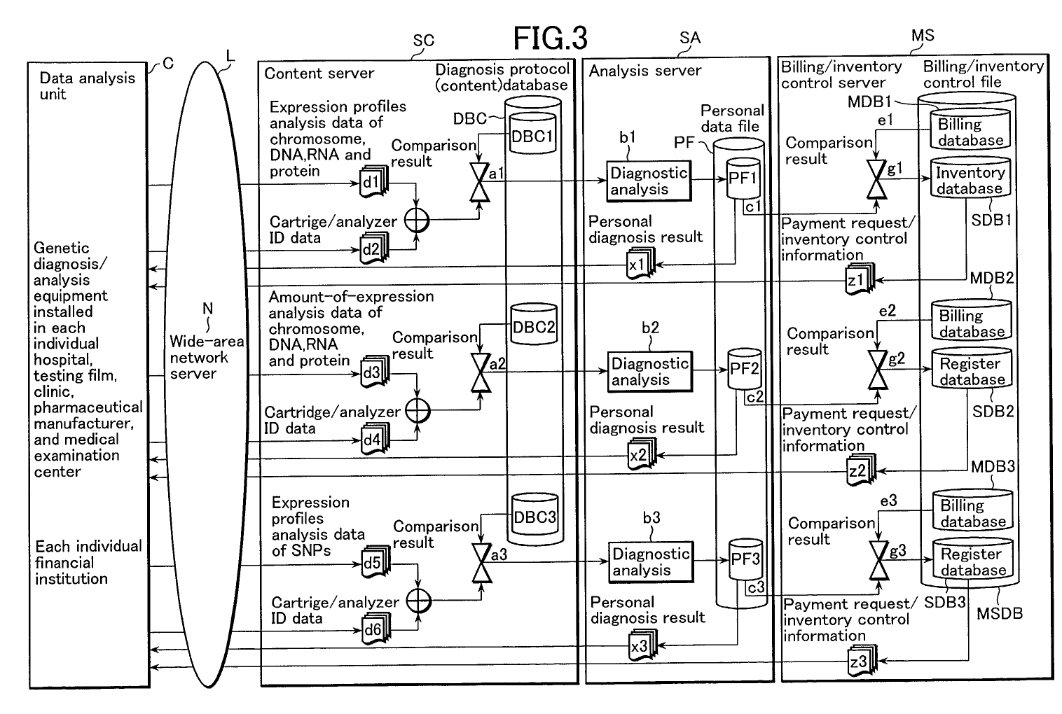

FIG. 3 is another block diagram showing the configuration of a medical data processing system according to the present invention.

In FIG. 3, a data analysis unit C is a small-scale computer system, such as a regular personal computer, installed to be accessible to, for example, a hospital, testing firm, clinic, doctor, pharmacist, pharmaceutical manufacturer, medical examination center, medical consultant, and financial institution, and is connected to a communications network L. The data analysis unit C is used as, for example, genetic diagnosis/analysis equipment.

Also connected to the communications network L through a wide-area network server N are a content server SC, analysis server SA, and billing/inventory control server MS that compose the medical data processing system according to the present invention.

The content server SC has the function for receiving data sent from the data analysis unit C and comparing the data with those in a database DBC that stores disease condition, disease names, treatment methods, medication methods and other information appropriate for different items of data as diagnosis protocols.

For example, the content server SC receives from the data analysis unit C the expression profiles analysis data dl of chromosome/DNA/RNA/protein or the cartridge/analyzer ID data d2 thereof, the amount-of-expression analysis data d3 of chromosome/DNA/RNA/protein or the cartridge/analyzer ID data d4 thereof, or the expression profiles analysis data d5 of SNPs or the cartridge/analyzer ID data d6 thereof.

The content server SC then compares the received data with various diagnosis protocols in databases DBC1, DBC2 and DBC3 that compose the database DBC, and outputs comparison results a1, a2 and a3, such as disease condition, disease names, treatment methods, and medication methods, to the analysis server SA.

Note that the system may be configured so that disease condition, disease names, treatment methods, medication methods and other information corresponding to the expression profiles analysis data dl of chromosome/DNA/RNA/protein or the cartridge/analyzer ID data d2 thereof, the amount-of-expression analysis data d3 of chromosome/DNA/RNA/protein or the cartridge/analyzer ID data d4 thereof, or the expression profiles analysis data d5 of SNPs or the cartridge/analyzer ID data d6 thereof can be registered with the content server SC as new content (diagnosis protocols) from each hospital, testing firm, clinic, doctor, pharmacist, pharmaceutical manufacturer, and medical examination center.

Next, the transmitted comparison results a1, a2 and a3 are handled as diagnostic analyses b1, b2 and b3 for correlation with each individual patient at the analysis server SA, and are registered as diagnostic analyses b1, b2 and b3 with information files PF1, PF2 and PF3 that compose a personal data file PF.

In that case, personal diagnosis results x1, x2 and x3 obtained as the result of matching the diagnostic analyses b1, b2 and b3 with the personal files PF1, PF2 and PF3 may be sent back to the data analysis unit C upon request from the data analysis unit C.

Furthermore, personal objective raw analysis data may be directly transmitted to the personal data file PF of the analysis server SA and stored therein from the data analysis unit C, without passing the data through the content server SC.

In a further step, personal information c1, c2 and c3 that contain diagnostic analyses b1, b2 and b3 are transmitted to the billing/inventory control server MS that has a billing/inventory control file MSDB. Then, the billing/inventory control server MS reads fee information e1, e2 and e3 corresponding to the comparison results a1, a2 and a3 (e.g., medication methods and treatment methods) obtained at the content server SC and contained in the personal information c1, c2 and c3, from billing databases MDB1, MDB2 and MDB3.

Comparison results g1, g2 and g3 thus obtained are checked with inventory/register databases SDB1, SDB2 and SDB3 that store stock data on corresponding medicines, medical treatment equipment and other things. Then, payment request/inventory control information z1, z2 and z3 (e.g., payment transaction information for each financial institution and information on the replenishment of medicines, medical treatment equipment and other things) are sent back to the data analysis unit C (each hospital, testing firm, clinic, doctor, pharmacist, pharmaceutical manufacturer, and medical examination center).

Note that the wide-area network server N is a block responsible for the core part of the process of data exchange between the data analysis unit C and the medical data processing system according to the present invention.

With the system thus configured, it is possible to 1) transmit the expression profiles analysis data dl of chromosome/DNA/RNA/protein or the cartridge/analyzer ID data d2 thereof, the amount-of-expression analysis data d3 of chromosome/DNA/RNA/protein or the cartridge/analyzer ID data d4 thereof, or the expression profiles analysis data d5 of SNPs or the cartridge/analyzer ID data d6 thereof, from the data analysis unit C of each hospital, testing firm, clinic, doctor, pharmacist, pharmaceutical manufacturer, medical examination center or other users to the content server SC in order to obtain the comparison results a1, a2 and a3, 2) obtain personal information c1, c2 and c3 from the analysis server SA according to these comparison results a1, a2 and a3, and 3) send back payment request/inventory control information z1, z2 and z3 (e.g., payment transaction information for each financial institution and information on the replenishment of medicines, medical treatment equipment and other things) to the data analysis unit C according to the personal information c1, c2 and c3.

Although the data analysis unit C (each hospital, testing firm, clinic, doctor, pharmacist, pharmaceutical manufacturer, and medical examination center) is configured so as to acquire so-called genetic diagnosis data, such as disease condition, disease names, treatment methods, and medication methods, corresponding to the aforementioned data d1, d2, d3 and d4 related to chromosome/DNA/RNA/protein and data d5 and d6 related to SNPs, these genetic data may be incorporated in an electronic clinical chart system on the side of the data analysis unit C.

In other words, there is a case that on the side of the data analysis unit C, the user performs regular tests, such as biochemical tests, blood tests, and urine analyses, upon patients, in addition to the aforementioned genetic diagnoses, in order to obtain general test data. There is also a case that the user obtains a record of earlier diseases of the patient in question as general diagnosis data and holds a collection of these data in the form of an electronic system.

In the present invention, an interface is incorporated in order to link the data analysis unit C to an electronic system built in each of the aforementioned hospital, testing firm, clinic, doctor, pharmacist, pharmaceutical manufacturer, and medical examination center.

With this system configuration, it is possible for the user on the side of the data analysis unit C (each hospital, testing firm, clinic, doctor, pharmacist, pharmaceutical manufacturer, and medical examination center) to combine the aforementioned general test data, general diagnosis data, and genetic diagnosis data provided by the system of the present invention, according to personal information, and thereby build a system of electronic data (electronic clinical charts).

As a summary of the discussion made heretofore, the following advantages are provided according to the present invention.

According to the medical data processing system of the present invention, it is possible to study and search for disease condition, disease names, treatment methods, medication methods and other information. It is also possible to acquire information on the inventory of medicines, medical treatment equipment and other things, and build a system whereby it is possible to smoothly and efficiently follow the procedure of paying expenses necessary for these processes.

Also according to the medical data processing system of the present invention, it is possible to electronically study and search for stock information through a database of medicines, medical treatment equipment and other things, in addition to studying and searching for disease condition, disease names, treatment methods, medication methods and other information. Thus, it is possible to prevent so-called erroneous diagnosis, including human errors in diagnosis and research.

Furthermore, according to the medical data processing system of the present invention, obtained genetic diagnosis data can be combined with general test data and general diagnosis data in order to build a system of electronic data (electronic clinical charts) and manage all patient-related medical data in a centralized manner.

What is claimed is:

1. A medical data processing system in which data analysis units for medical use are connected to a communications network, wherein a database of at least one case, whose corresponding recipes are stored as data and therefore a plurality of recipe items is provided, is installed as a portal site to be accessed by said data analysis units.

2. The medical data processing system of claim 1, wherein said case is subdivided into subdivided disease condition so as to have a hierarchical structure.

3. The medical data processing system of claim 1 or 2, wherein said portal site comprises a billing system for requesting accessees to execute billing according to the condition of use of said recipe items when access is made thereto.

4. The medical data processing system of claim 1, 2 or 3, wherein said recipe items are such that said corresponding recipes contained therein are linked to the location of storage of original data.

5. The medical data processing system of claim 1, 2 or 3, wherein said data analysis units gain access to said recipe items and download data contained therein.

6. A medical data processing system in which data analysis units for medical use are connected to a communications network, comprising:
a content server having databases for storing disease condition, disease names, treatment methods, medication methods, and other information, as diagnosis protocols, in order to output the result of comparing said databases with data sent from said data analysis unit;
an analysis server for correlating said comparison result with an individual patient as a diagnostic analysis and outputting personal information; and
a billing/inventory control server for outputting fee information appropriate for said comparison result included in said personal information from a billing database and sending back payment request/inventory control information to said data analysis units by outputting inventory control information from an inventory/register database containing stock data on, for example, medicines and treatment equipment.

7. The medical data processing system of claim 6, wherein said data is the expression profiles analysis data of chromosome/DNA/RNA/protein or the cartridge/analyzer ID data thereof, the amount-of-expression analysis data of chromosome/DNA/RNA/protein or the cartridge/analyzer ID data thereof, or the expression profiles analysis data of SNPs or the cartridge/analyzer ID data thereof.

8. The medical data processing system of claim 6 or 7, wherein disease condition, disease names, treatment methods, medication methods, and other information appropriate for said expression profiles analysis data of chromosome/DNA/RNA/protein or the cartridge/analyzer ID data thereof, said amount-of-expression analysis data of chromosome/DNA/RNA/protein or the cartridge/analyzer ID data thereof, or said expression profiles analysis data of SNPs or the cartridge/analyzer ID data thereof, from said data analysis units are registered as new content (diagnosis protocols) in said content server.

9. The medical data processing system of claim 6, wherein said analysis server receives personal objective raw analysis data directly sent from said data analysis units as personal information, and stores said information.

10. The medical data processing system of claim 6, wherein said data analysis units are provided with an interface for combining at least one piece of information, among said comparison result from said content server, said personal information from said analysis server, and said payment request/inventory control information from said billing/inventory control server, with regular test data or regular diagnosis data.
